(19) United States
(12) Patent Application Publication
(10) Pub. No.: US 2024/0197449 A1
(43) Pub. Date: Jun. 20, 2024

(54) METHODS AND APPARATUSES FOR MATCHING JAWS VIRTUAL DENTAL MODEL USING TEETH REFERENCE POINTS

(71) Applicant: Align Technology, Inc., San Jose, CA (US)

(72) Inventors: Mikhail STROGANOV, Moscow (RU); Mikhail GORODILOV, Novosibirsk (RU); Ksenia SHLAPAKOVA, Moscow (RU); Ilya PASTUSHKOV, Moscow (RU); Alexander YAROVENKO, Moscow (RU); Denis PARFENOV, Moscow (RU); Anna NESTEROVA, Moscow (RU); Danila CHESNOKOV, Moscow (RU); Georgiy SHUSHUEV, Moscow (RU); Sergei POPOV, Tambov (RU); Vasily PARAKETSOV, Moscow (RU)

(21) Appl. No.: 18/542,261

(22) Filed: Dec. 15, 2023

Related U.S. Application Data
(60) Provisional application No. 63/387,696, filed on Dec. 15, 2022.

(51) Int. Cl. A61C 13/00; A61C 9/00; G06T 7/00; G06T 7/11; G16H 70/00

(52) U.S. Cl. CPC A61C 13/0004; A61C 9/004; G06T 7/0014; G06T 7/11; G16H 70/00; G06T 2207/30036

(57) ABSTRACT

Methods and apparatuses to determine the best (e.g., closest) match between a patient's current or jaw configuration and one actual or intended jaw configurations from a series of jaw configurations for the same patient that were previously generated. These methods and/or apparatuses may be used to transform a prior tooth model, from a prior stage or stages of a treatment plan, into a current tooth arrangement. This may include revising the digital model.

### CLAIM OF PRIORITY

This patent application claims priority to U.S. Provisional Patent Application No. 63/387,696, titled “METHODS AND APPARATUSES FOR MATCHING JAWS VIRTUAL DENTAL MODEL USING TEETH REFERENCE POINTS,” filed Dec. 15, 2022, herein incorporated by reference in its entirety.

### BACKGROUND

Orthodontic procedures typically involve repositioning an individual's teeth to a desired arrangement in order to correct malocclusions and/or improve aesthetics. To achieve these objectives, orthodontic appliances such as braces, shell aligners, and the like can be applied to the individual's teeth by an orthodontic practitioner and/or by the individuals themselves. The appliance can be configured to exert force on one or more teeth in order to effect desired tooth movements according to a treatment plan.

Orthodontic aligners may include devices that are removable and/or replaceable over the teeth. Orthodontic aligners may be provided as part of an orthodontic treatment plan. In some orthodontic treatment plans involving removable and/or replaceable aligners, an individual may be provided plurality of orthodontic aligners over the course of treatment to make incremental position adjustments to the individual's teeth. These incremental adjustments may result in an overall change in the individual jaw including the teeth.

Some orthodontic aligners make use of a 3D model of the patient's teeth for treatment planning and tracking. The 3D modeling process can include scanning the patient's teeth with an intraoral scanner, generating a 3D model from the scanned data, and segmenting the 3D model to identify individual teeth and/or other intraoral features such as gingiva. Segmentation of 3D models is a complex computational process which can include separating teeth anatomy from gingiva and removing extra material and distortions from the scan. The result of the segmentation significantly affects treatment quality, and poor segmentation results can cause aligner fit issues, pain, and other customer complaints. To improve segmentation outcomes, automatically segmented scans can be manually reviewed and corrected by dedicated person, such as a DDT CAD designer, who can spend time to review and correct segmented dental models, further adding time and expense to orthodontic treatments.

There is a need for accurate, automated segmentation of scans of patients who previously were treated (“primary orders”) and have now been scanned again.

### SUMMARY OF THE DISCLOSURE

The methods and apparatuses described herein may simplify, improve and/or may automate manufacturing of aligners which are produced according to digital treatment plan. In particular, these methods and apparatuses may leverage a prior (e.g., earlier) treatment plan from the same patient in preparing a new or continued treatment plan. The prior (e.g., earlier) treatment plan may be a primary order treatment plan (“primary order”) and may include a sequence of teeth positions starting from the initial position the teeth and jaw(s) as original scanned from the patient. This primary order may be segmented, meaning that the oral scan may be divided into regions of identified teeth and/or gingival structures. The primary order treatment plan may end (e.g., with the final stage(s) of the primary treatment plan) so that the teeth are in a position required by the dental professional (e.g., doctor, orthodontist, etc.) as the goal of the treatment. The number of the position in the sequence of the treatment plan may be referred to as the treatment stage. In some examples, each stage may include a corresponding aligner for incrementally moving the subject's teeth during that stage of the treatment plan.

As described above, in some cases the subject (e.g., patient) has already completed a treatment plan and/or has stopped or suspended the treatment plan before completion, but the patient would benefit from and/or has requested a secondary order in order to further move or adjust the position of the patient's teeth. However, the treatment primary order treatment plan may no longer correspond to the subject's actual tooth position. For example the patient's actual tooth position may be different from the initial position, the final position and/or any of the intermediate stage positions. In these cases, it would be beneficial to use the primary order treatment plan to generate the secondary order treatment plan, and to produce an additional sequence of aligners (of the secondary order treatment plan) to support or correct previous treatment results.

In some examples, processing of secondary order treatment plans may beneficially reuse some treatment features from any previous order treatment plan (e.g., one or more of teeth surfaces, teeth numbers, attachments, final teeth position, etc.). Thus, in some examples, the secondary order treatment plan generation may be started from a scan of current patient's oral state; this scan may be segmented, including automatically segmented, and may be used to correct some inaccuracies of the segmentation by reusing segmentation from a primary order. Furthermore, in general the current model (digital model) based on the scan of the patient's current oral state may be matched to the jaws and teeth from the primary order and this match may be used to generate the secondary order treatment plan (or multiple potential second order treatment plans). In some cases after treatment has stopped or been suspended, the patient's teeth may remain in a state that is close to one of the treatment stages of primary order treatment plan. Thus, the methods and apparatuses described herein may automatically find the stage from the primary order treatment plan that best matches to the current teeth state and that combines features of the primary order state with the new secondary order state.

For example, described herein are methods apparatuses (e.g., devices, systems, etc. including software, hardware and firmware, such as a computing device readable medium having instructions stored thereon that are executable by a processor to cause a computing device to perform any of the methods described herein) that may match an arrangement of teeth of one or both jaws from a primary order (e.g., primary order treatment plan) to the teeth of a jaw or jaws of a scan of the patient's jaw or jaws before or as part of process for forming a secondary order (e.g., a secondary order treatment plan). In some examples the methods and apparatuses may match a jaw (e.g., an arrangement of teeth of a jaw) of a primary order with segmented teeth crowns from a scan (or scans, such as intraoral scans) of the same patient, for whom a secondary order treatment plan is desired. The methods and apparatuses described herein may find the closest treatment stage from previous (e.g., primary) order and may be used to indicate how to transform the closes treatment stage of the previous order so that it will fit or match the arrangement of teeth from the secondary order scan. In general these methods and apparatuses may include matching, manipulation and/or analysis of three-dimensional (3D) surfaces, use of one or more 3D scan(s), segmentation of a digital model based on the 3D scan(s). In some examples the methods and apparatuses described herein may use rigid transformation. Any of these methods and apparatuses may be used to generate one or more orthodontic appliances, and in particular, orthodontic appliances that are configured as part of a series or sequence of appliances (e.g., aligners, series or aligners, etc.).

The methods described herein may identify the best match between a patient's current jaw arrangement (e.g., tooth arrangement) and a stage of a prior (e.g., primary) treatment plan. In any of these methods and apparatuses point-to-point matching may be performed by obtaining reference points for the matching of elements of the jaws or teeth between the new (or more recent scan) and the primary order stages. In general, these methods and apparatuses may mitigate the effect of local teeth surfaces differences on the result of the matching, even where the patient's teeth and/or jaw may be different in morphology and even tooth count, due to extraction, wear, orthodontic procedures (e.g., fillings, caps, etc.), and the presence or absence of attachments on the teeth.

For example, described herein are methods of transforming a current digital model of a patient's dental arch using a prior treatment plan having a plurality of stages. Any of these methods may include: using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth; setting, for each matched pair, one or more reference points on the surface of each tooth of the prior treatment plan and mapping the one or more reference points to the surface of each tooth of the current digital model of the matched pair; comparing, for each stage, an arrangement of the matched pairs of teeth using the one or more reference points to identify which stage of the plurality of stages of the prior treatment plan best matches the current digital model; and outputting the stage of the plurality of stages of the prior treatment plan that best matches the current digital model as a new current digital model.

Any of these methods may include confirming a quality of match for each matching pair and rejecting each matching pair if the quality of match is below a threshold. For example, a quality of match for each matching pair may be confirmed by comparing crown regions of the prior treatment plan tooth and the matched current digital model tooth and rejecting each matching pair if the quality of match is below a threshold.

Any of these methods and apparatuses may include generating a tooth-to-crown transformation for each prior treatment plan tooth that is matched with a tooth of the current digital model. The prior treatment plan that best matches the current digital model may be transformed using the tooth-to-crown transformation to form the new current digital model (for output).

Comparing the arrangement of the matched pairs of teeth may comprise determining a minimum sum of squared distances between the reference points of the set of reference points for each stage and the points of the set of reference points for the current digital model. In some examples, comparing the arrangement of the matched pairs of teeth comprises generating a transformation between a set of reference points for each stage and a set of reference points for the current digital model.

In any of the methods and apparatuses described herein, outputting may include transforming the stage of the plurality of stages of the prior treatment plan that best matches the current digital model using the transformation between the set of reference points for each stage and the set of reference points for the current digital model and outputting the transformed stage of the plurality of stages as the new current digital model.

The methods and apparatuses described herein may also confirm that the one or more reference points mapped to the surface of each tooth of the current digital model of the matched pair are not in a line. If, for any one of the teeth of the current digital model the reference points are arranged in a line, the method of apparatus may select new reference points and/or exit out of the method and may instead use another method.

Any of the methods and apparatuses described herein may also include confirming that the number of matching pairs of the prior treatment plan and the current digital model is greater than three. If the total number of matching pairs is 3 or less, the method or apparatus may instead use another method to determine which of the stages of the prior treatment plan (e.g., primary order treatment plan) best match the current jaw arrangement and/or adapt the best matching stage of the prior treatment plan to the current jaw arrangement.

In some examples, the methods described herein may include a step of receiving the current digital model of the patient's dental arch, and/or receiving the prior treatment plan having the plurality of stages. In some examples, these methods an apparatuses may be configured to segment the current digital model of the patient's dental arch, e.g., using a known segmentation method. In some examples the methods and apparatuses described herein may determine tooth numbering of the current digital model. For example, any of these methods an apparatuses may include numbering the teeth of the patient's dental arch.

For example, a method of transforming a current digital model of a patient's dental arch using a prior treatment plan having a plurality of stages may include: using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth; rejecting each matching pair if a quality of match is below a threshold; setting, for each matched pair, one or more reference points on the surface of each tooth of the prior treatment plan and mapping the one or more reference points to the surface of each tooth of the current digital model of the matched pair; comparing, for each stage, an arrangement of the matched pairs of teeth using the one or more reference points to identify which stage of the plurality of stages of the prior treatment plan best matches the current digital model, wherein comparing the arrangement of the matched pairs of teeth comprises generating a transformation between a set of reference points for each stage and a set of reference points for the current digital model; transforming the stage of the plurality of stages of the prior treatment plan that best matches the current digital model using the transformation between the set of reference points for each stage and a set of reference points for the current digital model to form a new current digital model; and outputting the new current digital model.

Also described herein are apparatuses, including systems and software (e.g., configured as a non-transitory computing device readable medium having instructions stored thereon that are executable by a processor to cause a computing device to perform the steps of the computer-implemented method) configured to perform any of these methods, including all or some of these steps described herein. A processor may include hardware that runs the computer program code. Specifically, the term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

For example, a system for transforming a current digital model of a patient's dental arch using a prior treatment plan having a plurality of stages may include: one or more processors; a memory coupled to the one or more processors, the memory storing computer-program instructions, that, when executed by the one or more processors, perform a computer-implemented method comprising: using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth; setting, for each matched pair, one or more reference points on the surface of each tooth of the prior treatment plan and mapping the one or more reference points to the surface of each tooth of the current digital model of the matched pair; comparing, for each stage, an arrangement of the matched pairs of teeth using the one or more reference points to identify which stage of the plurality of stages of the prior treatment plan best matches the current digital model; outputting the stage of the plurality of stages of the prior treatment plan that best matches the current digital model as a new current digital model.

Any of these systems may be configured to perform any of the steps of the methods described above. For example, the systems described herein may be configured to confirm a quality of match for each matching pair and rejecting each matching pair if the quality of match is below a threshold. In some examples, the system may confirm a quality of match for each matching pair by comparing crown regions of the prior treatment plan tooth and the matched current digital model tooth and rejecting each matching pair if the quality of match is below a threshold. Any of these systems may generate a tooth-to-crown transformation for each prior treatment plan tooth that is matched with a tooth of the current digital model.

As described above, comparing the arrangement of the matched pairs of teeth may include determining a minimum sum of squared distances between the reference points of the set of reference points for each stage and the points of the set of reference points for the current digital model. Comparing the arrangement of the matched pairs of teeth may comprise generating a transformation between a set of reference points for each stage and a set of reference points for the current digital model.

Outputting may further comprise transforming the stage of the plurality of stages of the prior treatment plan that best matches the current digital model using the transformation between the set of reference points for each stage and the set of reference points for the current digital model and outputting the transformed stage of the plurality of stages as the new current digital model. As mentioned above, the system described herein may also confirm that the one or more reference points mapped to the surface of each tooth of the current digital model of the matched pair are not in a line, and/or may confirm that the number of matching pairs of the prior treatment plan and the current digital model is greater than three.

For example, a system for transforming a current digital model of a patient's dental arch using a prior treatment plan having a plurality of stages may include: one or more processors; a memory coupled to the one or more processors, the memory storing computer-program instructions, that, when executed by the one or more processors, perform a computer-implemented method comprising: using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth; rejecting each matching pair if a quality of match is below a threshold; setting, for each matched pair, one or more reference points on the surface of each tooth of the prior treatment plan and mapping the one or more reference points to the surface of each tooth of the current digital model of the matched pair; comparing, for each stage, an arrangement of the matched pairs of teeth using the one or more reference points to identify which stage of the plurality of stages of the prior treatment plan best matches the current digital model, wherein comparing the arrangement of the matched pairs of teeth comprises generating a transformation between a set of reference points for each stage and a set of reference points for the current digital model; transforming the stage of the plurality of stages of the prior treatment plan that best matches the current digital model using the transformation between the set of reference points for each stage and a set of reference points for the current digital model to form a new current digital model; and outputting the new current digital model.

Also descried herein are non-transitory computing device readable media having instructions stored thereon that are executable by a processor to cause a computing device to: a memory coupled to the one or more processors, the memory storing computer-program instructions, that, when executed by the one or more processors, perform a computer-implemented method comprising: use tooth numbering to form, for each stage of a prior treatment plan having a plurality of stages, matching pairs of teeth of the prior treatment plan and a current digital model of a patient's dental arch, wherein the prior treatment plan comprises a plurality of stages that each include a digital model of a dental arch configuration having a plurality of numbered teeth, further wherein the current digital model of the patient's dental arch comprises a plurality of numbered teeth; set, for each matched pair, one or more reference points on the surface of each tooth of the prior treatment plan and map the one or more reference points to the surface of each tooth of the current digital model of the matched pair; compare, for each stage, an arrangement of the matched pairs of teeth using the one or more reference points to identify which stage of the plurality of stages of the prior treatment plan best matches the current digital model; output the stage of the plurality of stages of the prior treatment plan that best matches the current digital model as a new current digital model.

A non-transitory computing device readable medium having instructions stored thereon that are executable by a processor to cause a computing device to: a memory coupled to the one or more processors, the memory storing computer-program instructions, that, when executed by the one or more processors, perform a computer-implemented method comprising: use tooth numbering to form, for each stage of a prior treatment plan having a plurality of stages, matching pairs of teeth of the prior treatment plan and a current digital model of a patient's dental arch, wherein the prior treatment plan comprises a plurality of stages that each include a digital model of a dental arch configuration having a plurality of numbered teeth, further wherein the current digital model of the patient's dental arch comprises a plurality of numbered teeth; reject each matching pair if a quality of match is below a threshold; set, for each matched pair, one or more reference points on the surface of each tooth of the prior treatment plan and map the one or more reference points to the surface of each tooth of the current digital model of the matched pair; compare, for each stage, an arrangement of the matched pairs of teeth using the one or more reference points to identify which stage of the plurality of stages of the prior treatment plan best matches the current digital model, wherein comparing the arrangement of the matched pairs of teeth comprises generating a transformation between a set of reference points for each stage and a set of reference points for the current digital model; transform the stage of the plurality of stages of the prior treatment plan that best matches the current digital model using the transformation between the set of reference points for each stage and a set of reference points for the current digital model to form a new current digital model; and output the new current digital model.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1C illustrate an example of a treatment plan including a plurality of stages. Each stage includes a segmented and numbered three-dimensional (3D) digital model an arrangement of teeth in a jaw (e.g., upper arch). FIG. 1A shows a first stage, FIG. 1B shows an intermediate stage, and FIG. 1C shows a final stage of the treatment plan.

FIG. 2A shows examples of a digital model of a current (e.g., secondary) digital model of the patient's dental arch.

FIG. 2B shows a merged view of the best matching stage of a prior treatment plan and the current digital model of the patient's dental arch, showing regions of overlap and non-overlap.

FIG. 3 schematically illustrates a method of identifying matching pairs between a prior treatment plan and a current digital model of the patient's dental arch.

FIG. 4 schematically illustrates a method of generating a tooth-to-crown transformation between a prior treatment plan tooth that is matched with a tooth of the current digital model.

FIG. 5 schematically illustrates a method of identifying setting reference points on the teeth of the prior treatment plan that are matched with a tooth of the current digital model, and translating these reference points to the matched teeth of the current digital model.

FIG. 6 schematically illustrates comparison between the stages of the prior treatment plan (primary order teeth stages, on left) and the current digital model (right).

FIG. 7 schematically illustrates the comparison, for each stage, of the arrangement of the matched pairs of teeth using the one or more reference points to identify which stage of the plurality of stages of the prior treatment plan best matches the current digital model.

FIG. 8 schematically illustrates an example of a method of transforming a current digital model of a patient's dental arch using a prior treatment plan having a plurality of stages.

FIG. 9 is a diagram illustrating one variation of a dental computing environment including or incorporating a system for transforming a current digital model of a patient's dental arch using a prior treatment plan having a plurality of stages as described herein.

### DETAILED DESCRIPTION

Described herein are methods and apparatuses that determine the best (e.g., closest) match between a patient's current or jaw configuration and one actual or intended jaw configurations from a series of jaw configurations for the same patient that were previously generated. As used herein a jaw configuration may include the arrangement of teeth in the jaw or jaws (e.g., upper and/or lower jaws). The series of jaw configurations for the same patient that were previously generated may form a prior orthodontic treatment plan. The prior orthodontic treatment plan may be referred to equivalently as a primary order. The methods and apparatuses described herein may be particularly advantageous compared to other matching methods, as they may provide more precise results because they do not require that the same number of teeth be present in the jaw configurations being compared, and because they do not rely or require segmentation of the particular tooth or teeth within the jaw configurations being compared. Furthermore these methods and apparatuses may have a much lower computational complexity, and may require fewer computational cycles and processing. Thus, they may be performed locally or remotely very quickly, while still providing highly accurate results. This may be achieved, at least in part, by applying the techniques described herein to identify a small set of points (in some cases a minimal set) from the jaw configurations (e.g., digital scans, models or images of the jaw configurations), and comparing the resulting small set of points of across multiple different jaw configurations. These techniques may be particular advantageous as compared to other systems or techniques that perform matching based on matching of surfaces. The methods and apparatuses described herein therefore represent an improvement in the operation of these systems.

For example, the methods and apparatuses described herein may be used to identify which treatment stage of a prior treatment plan (e.g., a primary order treatment plan) best matches a particular configuration of a patient's jaw, and in particular, which treatment stage best matches a current configuration of a patient's jaw, as determined based on a recent or current scan of the patient's dentition. These methods and apparatuses may identify a transformation between the current configuration of the patient's jaws (e.g., teeth) and the configuration or position of the patient's teeth from a primary order treatment plan, so that after applying the transformation to the primary order, e.g., the jaw configuration of the various stages of a primary order treatment plan, in which the teeth crowns of each stage of the primary order of the treatment plan approximate much of the position and orientations of the crows of the teeth in the patient's current jaw configuration.

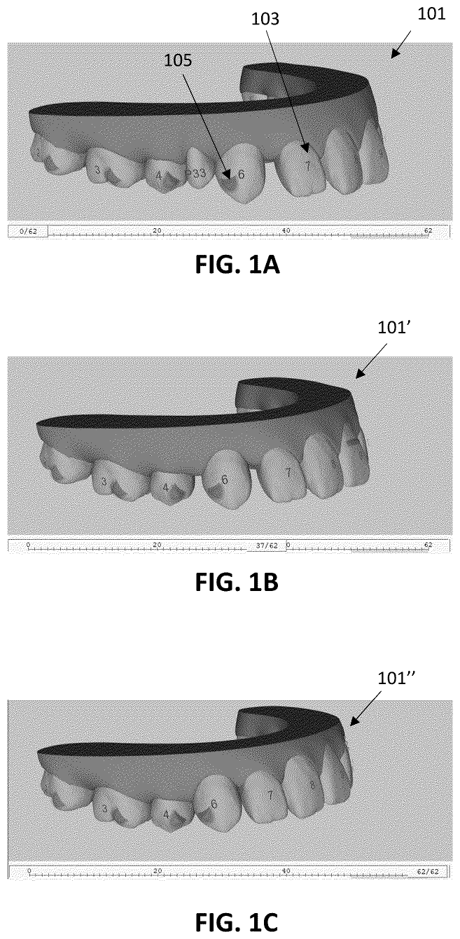

In any of these methods and apparatuses, a primary order treatment plan may be represented by the sequence of teeth positions starting from an initial position that may be segmented from an oral scan, and finishing in a final position, as required by the doctor as the goal of the treatment. For example, FIGS. 1A-1C illustrate examples of digital models 101, 101′, 101″ of a series of digital models of a patient's teeth starting with an initial position 101 in which the teeth have been segmented and numbered 103, shown in FIG. 1A. In FIGS. 1A-1C 62 the treatment plan includes 62 stages, in which teeth are gradually repositioned. FIGS. 1B and 1C shows subsequent stages, including a middle stage (FIG. 1B showing stage 37) and the final position (FIG. 1C, showing stage 62). In this example, the digital models represent accurate models of the patient's teeth as original scanned. Attachments 105 have also been shown or included on the teeth.

In some cases the original treatment plan shown by the representative digital models in FIGS. 1A-1C may be completed and the teeth may relapse or may continue to move. In some cases the treatment may be incomplete or interrupted for some reasons. In any event, a second treatment, referred to herein as a secondary order or as a secondary order treatment plan may be provided by initially taking a second scan of the patient's dentition or otherwise generating a digital model of the patient's actual tooth position. The new (current) tooth scan may be used as an input the method and apparatuses described herein. The current tooth scan may be automatically segmented so that the crowns of the teeth are identified from the scan. The crowns may be automatically or semi-automatically (or manually) numbered, and may be numbered using any appropriate numbering scheme. Segmenting and/or numbering may be performed as part of the methods and apparatuses described herein, or they may be segmented and/or numbered beforehand. The segmented and numbered current tooth scan may be referred to as a secondary scan.

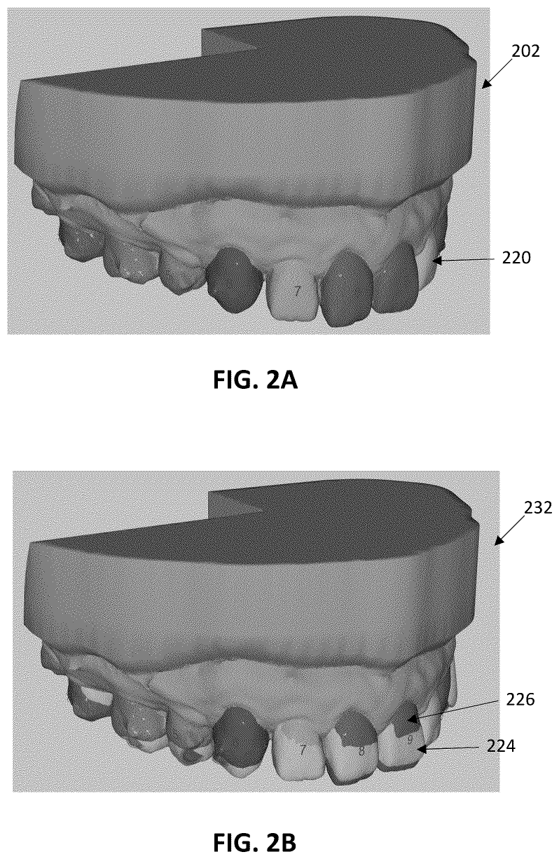

In general, these methods and apparatuses may output an indicator of which stage of the primary order treatment plan best matches the current configuration of the patient's teeth. In some examples, the output includes a transformation of the stage of the primary order jaw and teeth such that teeth of primary order will fit crowns of secondary one. In some examples, this may include the position of the patient's teeth among each other remains from one of the stages of primary order treatment plan. In some examples this may include an indicator of how well the current position(s) of the patient's teeth (or one or more individual teeth) fits or matches the best-fit stage from the primary order. The output may include a model or image showing how well the current position(s) of the patient's teeth fit or matches the best-fit stage from the primary order. For example, FIGS. 2A and 2B illustrate an example of an input and output for one example of a method (and apparatus) as described herein.

In FIG. 2A a digital model of the patient's current jaw configuration (for an upper jaw 202) that may be used as an input is shown, showing individual segmented teeth 220, each of which has been colored and numbered according to a standard tooth numbering system. This digital model is shown as a three-dimensional digital model of the patient's upper dental arch. In some examples, both the upper and lower dental arches may be processed together (and compared to primary orders having both upper and lower dental arches) or the upper and lower dental arches may be processed separately.

FIG. 2B illustrates one example of an output (or portion of an output) of the methods and apparatuses described herein. In this example the output 232 includes a three-dimensional digital model (or one or more images thereof) showing the treatment stage that best matches the current jaw configuration and overlap with the input current jaw configuration; the current jaw configuration is shown in color, as described above, and segmented. The common teeth are numbered. Overlapping regions are apparent as the colored and un-colored (or shaded and un-shaded) regions. For example, the tooth labeled “9” in FIG. 2B shows regions reflecting the current position of the tooth from the current jaw configuration 226 and regions that reflect the position of the corresponding tooth from the best matching stage of the primary order treatment plan 224.

In general, the output of the methods and apparatuses described herein may be a new current digital model. In some examples the output is primarily or exclusively a textual or alphanumeric output. For example, the output may be an indicator of the stage of the primary order treatment plan that best matches. In some examples this output may include an indicator (graphic, numeric score, etc.) for the quality of the match, as mentioned and illustrated above in FIG. 2B. In some examples the output may be a copy of the stage of the primary order treatment plan and/or any notations, comments, etc. (e.g., from the dental professional associated with the primary order). In some examples the output may be a description of the stage of the primary order treatment plan. In some examples, the output may be a new current digital model that is a transformed version of the closest match of the prior treatment plan stages, so that the teeth of the closest match of the prior treatment plan stage more closely (or exactly) match the position and/or orientation of the teeth of the current digital model of the patient's dental arch.

In general, the methods and apparatuses (e.g., systems and devices, including software), may be configured to form matching pairs of teeth between the teeth of the prior treatment plan and the teeth of the current digital model, to set corresponding reference points on the matching teeth, use these reference points to find the stage of the prior dental treatment plan that best matches the current arrangement of the patient's teeth, and determine a transform for transforming the best-matching stage to correspond to the current arrangement of the patient's teeth.

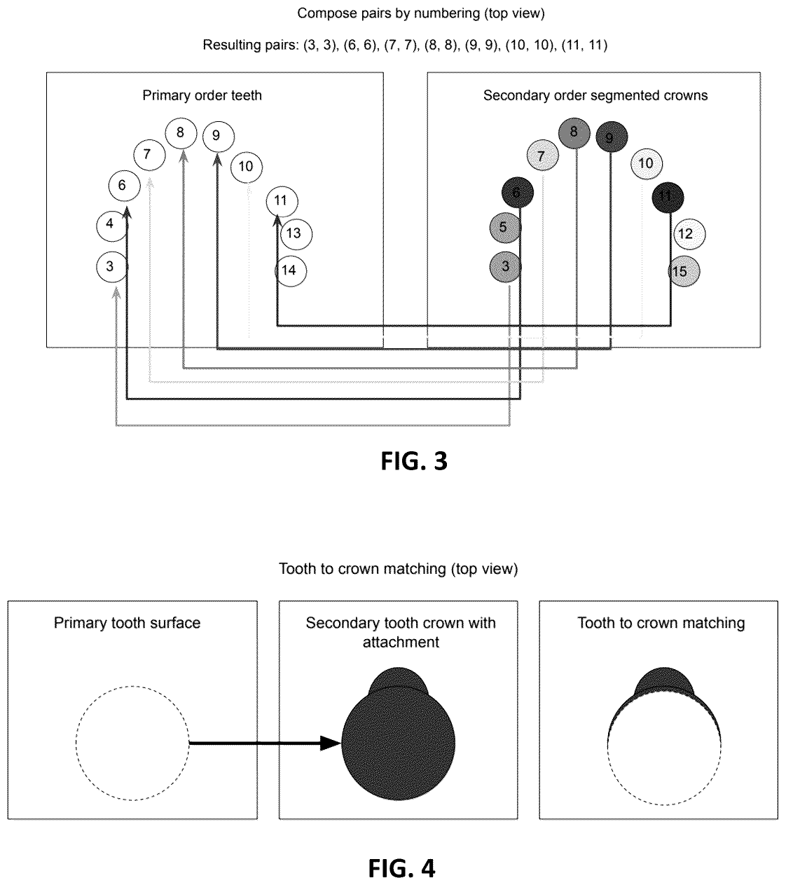

For example, FIG. 3 schematically illustrates one example of identifying matches between the teeth of a prior treatment plan (shown on the left) and the current arrangement of the patient's teeth (shown on the right). In general, matching pairs of teeth may be identified for each stage of the prior treatment plan with the current arrangement of the teeth. Matching may be made by matching tooth number, as shown in FIG. 3. For example, the teeth of the digital model for each stage of the prior treatment plan may be segmented and numbered. In some cases the segmentation and/or numbering of the prior treatment plan may be particularly reliable, as it may have been confirmed (manually, automatically and/or semi-automatically) during the original treatment plan preparation and treatment.

As shown in FIG. 3, matching pairs maybe formed consisting of a tooth from the prior treatment plan (e.g., primary order) and a tooth crown from the current arrangement of the patient's teeth (e.g., a secondary order) by matching tooth numbering. The tooth numbering of the current teeth may be determined automatically or semi-automatically either before or as part of the methods and apparatuses described herein. Thus, a plurality of tooth pairs may each include teeth having similarity segmented tooth numbering.

For each pair (each matching tooth pair), the method or apparatus may find a tooth-to-crown rigid transformation that matches the prior tooth surfaces (from the prior treatment plan tooth surface) with the current tooth (e.g., secondary order) crowns. The methods and apparatuses described herein may accommodate some differences between the prior tooth surface and current crown surface due to treatment features such as attachments, buttons, etc. For example, as shown in FIG. 4, the current teeth (“secondary tooth crown”) may include one or more treatment features such as attachments that may support tooth movement with required forces (e.g., for engaging with an aligner).

FIG. 4 illustrates the matching of the tooth surfaces between the prior dental plan (primary tooth surface) and the current (secondary) tooth crown. In any of the methods and apparatuses described herein a comparison between the matched teeth between the prior digital models of the tooth and the current digital model of the tooth may be made in order to confirm the matched pairs. In some examples, if the match is not sufficient (e.g., is outside of an acceptable range) then the match may be rejected. Thus, in some examples, the pairs may be filtered by the quality of the matching from previous step. In one example, a quality metric for the step might be a maximum distance between surfaces of the prior digital model and the current tooth digital model, and/or a proportion of matched area between the surface of the prior digital model and the current tooth digital model, etc.

In general, the methods and apparatuses described herein may be used where there are sufficient numbers of matched teeth between the prior treatment plan and the current digital model of the teeth. For example, these methods and apparatuses may include a threshold for proceeding with the method based on the number of matched; if the number of matched pairs is greater than two, greater than three, greater than four, etc. then the method may continue. If not (e.g., if the number of matched parrs is not greater than, for example, 3), then the apparatus may be configured to stop and use or suggest using a different technique, including a prior, more manual and/or processing intensive previous implementation.

As mentioned above, the output may be transformed based on one or more of: a tooth-to-crown transformation for each prior treatment plan tooth based on the matching tooth of the current digital model. Thus, all or some of the teeth (e.g., matching teeth) in the best matching of the prior treatment plan may be transformed to more closely align with the morphology of the current teeth. This is illustrated in FIG. 4. In this example, the tooth-to-crown matching (shown on the far left) illustrates a difference between the current tooth and the prior treatment plan tooth. This difference may be used as a tooth-to-crown transformation. Once the closest stage of the prior treatment plan has been identified, as described herein, the matching teeth (e.g., teeth for which there are matched pairs) of the prior treatment plan may be transformed to correspond to the matching current teeth using the tooth-to-crown transformation.

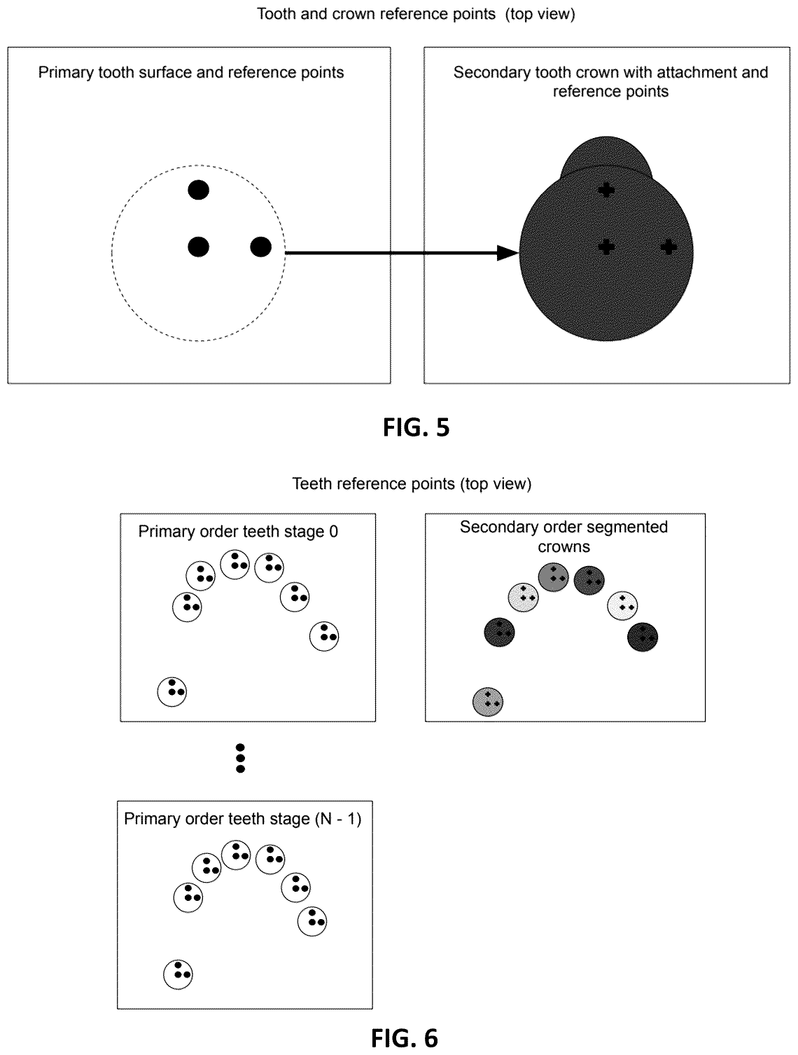

Following identification of matching teeth between the prior treatment plan and the current digital model of the teeth, reference points may be identified on the tooth crown and mapped between the prior treatment plan teeth (for each stage of the prior treatment plan) and the current teeth. For example, during this step, the methods and apparatus may make arrays of teeth reference points for each of the teeth that were matched. The tooth reference points may include a sequence of points in 3D space which are used to describe tooth surface or crown position and orientation. Any appropriate number of points may be used, preferably as small a number of points as possible, such as 3 points per tooth. The reference points may be placed or created on the tooth surface of the prior treatment plan tooth for each pair. For example, a crown center and two or more points shifted along x, y, z axes in tooth crown coordinate system may be used. Corresponding points on the digital model of the current tooth may then be identified. For example, points on the current tooth crown (“secondary order crown”) may be obtained as reference points for primary order surface transformed by tooth-to-crown transformation described above. This is illustrated in FIG. 5. Reference points may be identified in this manner for each tooth of the prior treatment plan, and applied across all of the N stages of the prior treatment plan.

For example, in FIG. 6, N arrays (one for each treatment stage of the prior treatment plan) of reference points are identified, and a single array for the current tooth arrangement (e.g., from the digital model of the current tooth arrangement). In FIG. 6, to obtain reference points for teeth for some stages, the initial reference points of the teeth may be transformed according to tooth position for this stage.

Once the reference points have been identified for each stage and for the current tooth arrangement, these reference points may be used to find the closet (e.g., “best”) match of the current tooth arrangement and a stage of the prior treatment plan. For each array of reference points of the N stages of the prior treatment plan, obtained as mentioned above, the methods and apparatuses described herein may calculate rigid transformation that minimizes the distance, e.g., that minimizes the sum of squared distances between all (or some) of the related pairs of reference points between the prior treatment plan stage and the current tooth arrangement.

In general these methods may determine a score for the comparison of the reference points. In some examples certain teeth and/or certain reference points may be weighted more or less (higher or lower), when calculating the score, such as when calculating a minimum of the sum of the squared distances. In some examples the score may be referred to as the error of the transformation between the prior tooth arrangement for each stage and the current tooth arrangement. The score (or error) may be any metric that reflects a distance between two sets of points, such as (but not limited to) a mean squared distance, mean absolute distance, maximum distance, etc. As mentioned individual points (or teeth) may be weighted when calculating the score/error. The best match of the stages of the prior treatment plan may be determined based on the comparison and the resulting score. These methods may be used to assign a weight to each reference point which may reduce the effect of some points on the resulting score. The weights might be used in computation of points matching transformation and in calculation of matching error. The weights may be assigned according to tooth-to-crown matching score or by some another approaches.

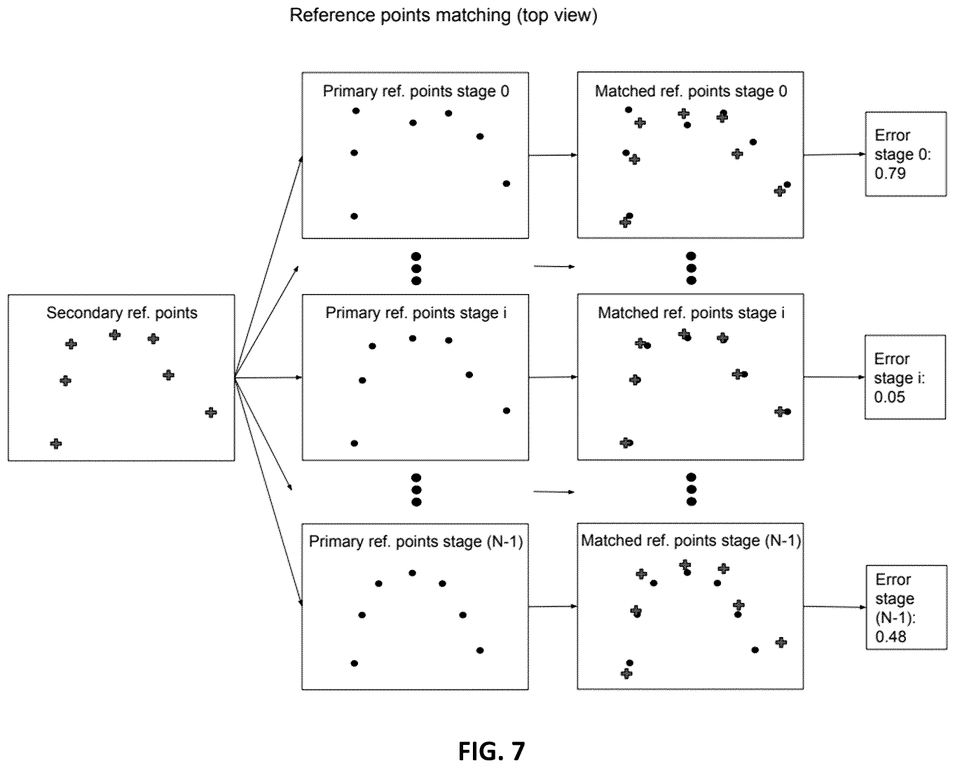

For example, as illustrated in FIG. 7, the lowest score, representing the lowest error between the prior treatment plan stage i and the current tooth arrangement (shown in this example as 0.05) may represent the closet match. The best match may be chosen from the score. In some examples a threshold may be applied when identifying a best match. For example, the score or error must be less than some threshold score. Once the best matching stage has been determined, a transformation, transforming the best arrangement of the best matching stage to more closely match the current tooth arrangement.

Thus, in any of these examples the method and apparatus may include a transformation of the tooth arrangement of the best matching treatment stage so that the teeth and tooth positions more closely match the tooth and tooth positions of the current tooth arrangement. As mentioned above, the individual teeth may also be transformed (e.g., using a tooth-to-crown transformation).

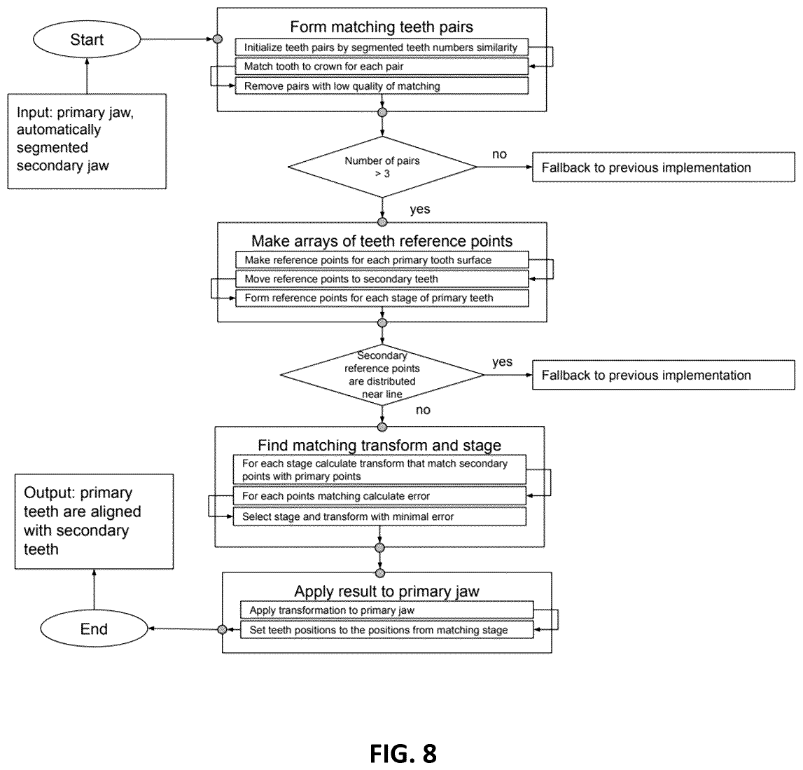

FIG. 8 is a flowchart illustrating the operation of a method or apparatus as described herein. In some examples, this method may show a method of matching a prior stage of a treatment plan to a current tooth arrangement (e.g., using a digital model) as described above. For example, in FIG. 8, the method or system may start by processing (e.g., receiving, recording, etc.) a prior treatment plan and a digital model of a current arrangement of a patient's teeth. Note that in any of these examples the term “current” may refer to a more current (e.g., subsequent) arrangement of the patient's teeth. Preferably this arrangement may reflect the actual arrangement of the patient's teeth at the present time.

As described above, matching tooth pairs may be identified between the teeth of the prior treatment plan and the current arrangement of the teeth. Once matched, the results of the match may be applied to the prior treatment plan stages, and the prior treatment plan teeth be used to generate reference points that may then been applied to the current teeth. The resulting reference points may then be used to find a best match between the current teeth arrangement and the prior treatment plan stages.

As described in FIG. 8, in some cases the methods and apparatuses may determine that the technique is not well suited to the data and may instead switch to, or indicate that, another technique should be used to estimate the best match between the prior treatment plan stages and the current arrangement of the teeth, e.g., an existing approach for jaw matching. For example, as described above, in some cases not enough teeth pairs (e.g., less than 3, less than 4, etc.) are matched. A small number of pairs may not reflect well the position of the jaw. In some examples the reference points on a tooth of the current tooth arrangement may be distributed in a line or near line. In this case, matching may not reflect accurate result for the jaw.

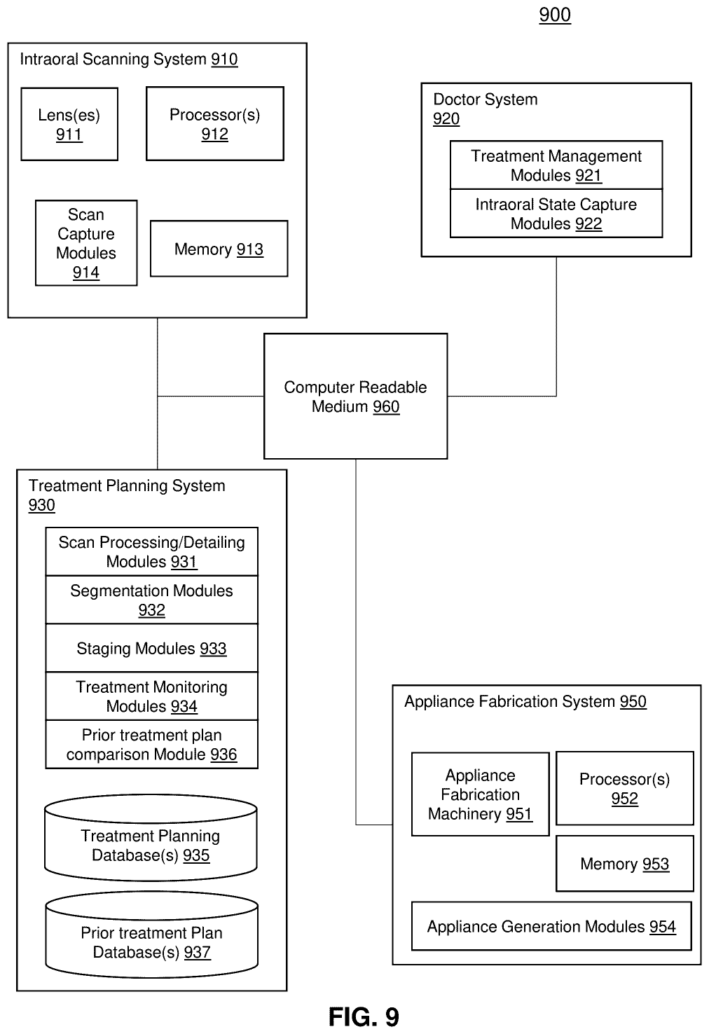

In general, the methods and apparatuses described herein for using a prior treatment plan to modify a treatment plan (e.g., staging of a treatment plan) may be used at one or more parts of a dental computing environment, including as part of an intraoral scanning system, a doctor system, a treatment planning system, and/or a fabrication system. In particular, these methods and apparatuses may be used as part of a treatment planning system, for example, to determine staging for treating a patient's teeth. For example, FIG. 9 is a diagram illustrating one variation of a dental computing environment 900 that may generate one or more orthodontic treatment plans specific to a patient, and fabricate dental appliances that may accomplish the treatment plan to treat a patient, under the direction of a dental professional. The example dental computing environment 900 shown in FIG. 9 includes an intraoral scanning system 910, a doctor system 920, a treatment planning system 930, an appliance fabrication system 950, and computer-readable medium 960. In some variations the dental computing environment (dental computing system) 900 may include just one or a subset of these systems (which may also be referred to as sub-systems of the overall system 900). Further, one or more of these systems may be combined or integrated with one or more of the other systems (sub-systems), such as, e.g., the treatment planning system 930 and the doctor system 920 may be part of a remote server accessible by a doctor interface. The computer readable medium 960 may divided between all or some of the systems (subsystems); for example, the treatment planning system 930 and appliance fabrication system 950 may be part of the same sub-system and may be on a computer readable medium 960. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, the intraoral scanning system 910 can include lens(es) 911, processor(s) 912, a memory 913, scan capture modules 914, and outcome simulation modules 915. In general, the intraoral scanning system 910 can capture one or more images of a patient's dentition. Use of the intraoral scanning system 910 may be in a clinical setting (doctor's office or the like) or in a patient-selected setting (the patient's home, for example). In some cases, operations of the intraoral scanning system 910 may be performed by an intraoral scanner, dental camera, cell phone or any other feasible device.

The lens(es) 911 include one or more lenses and optical sensors to capture reflected light, particularly from a patient's dentition. The scan capture modules 914 can include instructions (such as non-transitory computer-readable instructions) that may be stored in the memory 913 and executed by the processor(s) 99 to control the capture of any number of images of the patient's dentition.

As mentioned, in some examples the methods and apparatuses described herein for generating a 3D model including one or more teeth may be part of, or accessible by, the intraoral scanning system 910, computer readable medium 960 and/or treatment planning system 930.

Any of the component systems or sub-systems of the dental computing environment 900 may access or use the patient's prior treatment plan information to modify or form a current treatment plan or digital model or the patient's teeth as described by the methods and apparatuses described herein. For example, the doctor system 920 may include treatment management modules 921 and intraoral state capture modules 922 that may be used to modify a current digital model of the patient's teeth based on a prior treatment plan.

The treatment planning system 930 may include any of the methods and apparatuses described herein, and/or may transform a current digital model of a patient's dental arch using a prior treatment plan. The treatment planning system 930 may include scan processing/detailing modules 931, segmentation modules 932, staging modules 933, treatment monitoring modules 934, treatment planning database(s) 935, and prior treatment plan comparison modules 936. In general, the treatment planning system 930 can determine a treatment plan for any feasible patient. The scan processing/detailing modules 931 can receive or obtain dental scans (such as scans from the intraoral scanning system 910) and can process the scans to “clean” them by removing scan errors and, in some cases, enhancing details of the scanned image. The treatment planning system may also include a prior treatment plan database 937 including one or more prior treatment plans specific to the patient, which may be accessed by the prior treatment plan module and/or the doctor system and general treatment planning system.

The treatment planning system 930 may include a segmentation system that segments a model into separate components. For example, the treatment planning system 930 may include a segmentation modules 932 that can segment a dental model (such as a 3D dental model) into separate parts including separate teeth, gums, jaw bones, and the like. In some cases, the dental models may be based on scan data from the scan processing/detailing modules 931.

The staging modules 933 may determine different stages of a treatment plan. Each stage may correspond to a different dental aligner. In some examples, the staging modules 933 may also determine the final position of the patient's teeth, in accordance with a treatment plan. Thus, the staging modules 933 can determine some or all of a patient's orthodontic treatment plan. In some examples, the staging modules 933 can simulate movement of a patient's teeth in accordance with the different stages of the patient's treatment plan.

The treatment monitoring modules 934 can monitor the progress of an orthodontic treatment plan. In some examples, the treatment monitoring modules 934 can provide an analysis of progress of treatment plans to a clinician. The orthodontic treatment plans may be stored in the treatment planning database(s) 935. Although not shown here, the treatment planning system 930 can include one or more processors configured to execute any feasible non-transitory computer-readable instructions to perform any feasible operations described herein.

The prior treatment planning modules 936 can determine one or more stages of the plurality of stages of a prior treatment plan that best match the current digital model in order form a new current digital model.

The doctor system 920 may provide a “doctor facing” interface to the computing environment 900. The treatment management modules 921 can perform any operations that enable a doctor or other clinician to manage the treatment of any patient. In some examples, the treatment management modules 921 may provide a visualization and/or simulation of the patient's dentition with respect to a treatment plan. For example, the doctor system 920 may include a user interface for the doctor that allows the doctor to manipulate a 3D model and review the comparison between the prior treatment plan stage(s) and a current 3D model of the patient's teeth.

The intraoral state capture modules 922 can provide images of the patient's dentition to a clinician through the doctor system 920. The images may be captured through the intraoral scanning system 910 and may also include images of a simulation of tooth movement based on a treatment plan.

In some examples, the treatment management modules 921 can enable the doctor to modify or revise a treatment plan, including based on the prior treatment plan, as described above. The doctor system 920 may include one or more processors configured to execute any feasible non-transitory computer-readable instructions to perform any feasible operations described herein.

The appliance fabrication system 950 can include appliance fabrication machinery 951, processor(s) 952, memory 953, and appliance generation modules 954. In general, the appliance fabrication system 950 can directly or indirectly fabricate aligners to implement an orthodontic treatment plan. In some examples, the orthodontic treatment plan may be stored in the treatment planning database(s) 935.

The appliance fabrication machinery 951 may include any feasible implement or apparatus that can fabricate any suitable dental aligner. The appliance generation modules 954 may include any non-transitory computer-readable instructions that, when executed by the processor(s) 952, can direct the appliance fabrication machinery 951 to produce one or more dental aligners. The memory 953 may store data or instructions for use by the processor(s) 952. In some examples, the memory 953 may temporarily store a treatment plan, dental models, or intraoral scans.

The computer-readable medium 960 may include some or all of the elements described herein with respect to the dental computing environment 900. The computer-readable medium 960 may include non-transitory computer-readable instructions that, when executed by a processor, can provide the functionality of any device, machine, or module described herein.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, eaches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of transforming a current digital model of a patient's dental arch using a prior treatment plan having a plurality of stages, the method comprising:
using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth;
setting, for each matched pair, one or more reference points on a surface of each tooth of the prior treatment plan and mapping the one or more reference points to the surface of each tooth of the current digital model of the matched pair;
comparing, for each stage, an arrangement of the matched pairs of teeth using the one or more reference points to identify which stage of the plurality of stages of the prior treatment plan best matches the current digital model; and
outputting the stage of the plurality of stages of the prior treatment plan that best matches the current digital model as a new current digital model.

2. The method of claim 1, further comprising confirming a quality of match for each matching pair and rejecting each matching pair if the quality of match is below a threshold.

3. The method of claim 1, further comprising confirming a quality of match for each matching pair by comparing crown regions of the prior treatment plan tooth and the matched current digital model tooth and rejecting each matching pair if the quality of match is below a threshold.

4. The method of claim 1, further comprising generating a tooth-to-crown transformation for each prior treatment plan tooth that is matched with a tooth of the current digital model.

5. The method of claim 1, wherein comparing the arrangement of the matched pairs of teeth comprises determining a minimum sum of squared distances between the reference points of the set of reference points for each stage and the points of the set of reference points for the current digital model.

6. The method of claim 1, wherein comparing the arrangement of the matched pairs of teeth comprises generating a transformation between a set of reference points for each stage and a set of reference points for the current digital model.

7. The method of claim 6, wherein outputting further comprises transforming the stage of the plurality of stages of the prior treatment plan that best matches the current digital model using the transformation between the set of reference points for each stage and the set of reference points for the current digital model and outputting the transformed stage of the plurality of stages as the new current digital model.

8. The method of claim 1, further comprising confirming that the one or more reference points mapped to the surface of each tooth of the current digital model of the matched pair are not in a line.

9. The method of claim 1, further comprising confirming that the number of matching pairs of the prior treatment plan and the current digital model is greater than three.

10. The method of claim 1, further comprising receiving the current digital model of the patient's dental arch.

11. The method of claim 1, further comprising receiving the prior treatment plan having the plurality of stages.

12. The method of claim 1, further comprising segmenting the current digital model of the patient's dental arch.

13. The method of claim 1, further comprising numbering the teeth of the patient's dental arch.

14. A method of transforming a current digital model of a patient's dental arch using a prior treatment plan having a plurality of stages, the method comprising:
using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth;
setting, for each matched pair, one or more reference points on a surface of each tooth of the prior treatment plan and mapping the one or more reference points to the surface of each tooth of the current digital model of the matched pair;
comparing, for each stage, an arrangement of the matched pairs of teeth using the one or more reference points to identify which stage of the plurality of stages of the prior treatment plan best matches the current digital model; and
outputting the stage of the plurality of stages of the prior treatment plan that best matches the current digital model as a new current digital model.

15. The method of claim 14, further comprising confirming a quality of match for each matching pair and rejecting each matching pair if the quality of match is below a threshold.

16. The method of claim 14, further comprising confirming a quality of match for each matching pair by comparing crown regions of the prior treatment plan tooth and the matched current digital model tooth and rejecting each matching pair if the quality of match is below a threshold.

17. The method of claim 14, further comprising generating a tooth-to-crown transformation for each prior treatment plan tooth that is matched with a tooth of the current digital model.

18. The method of claim 14, wherein comparing the arrangement of the matched pairs of teeth comprises determining a minimum sum of squared distances between the reference points of the set of reference points for each stage and the points of the set of reference points for the current digital model.

19. The method of claim 14, wherein comparing the arrangement of the matched pairs of teeth comprises generating a transformation between a set of reference points for each stage and a set of reference points for the current digital model.

20. The method of claim 19, wherein outputting further comprises transforming the stage of the plurality of stages of the prior treatment plan that best matches the current digital model using the transformation between the set of reference points for each stage and the set of reference points for the current digital model and outputting the transformed stage of the plurality of stages as the new current digital model.

21. The method of claim 14, further comprising confirming that the one or more reference points mapped to the surface of each tooth of the current digital model of the matched pair are not in a line.

22. The method of claim 14, further comprising confirming that the number of matching pairs of the prior treatment plan and the current digital model is greater than three.

23. The method of claim 14, further comprising receiving the current digital model of the patient's dental arch.

24. The method of claim 14, further comprising receiving the prior treatment plan having the plurality of stages.

25. The method of claim 14, further comprising segmenting the current digital model of the patient's dental arch.

26. The method of claim 14, further comprising numbering the teeth of the patient's dental arch.

27. A method of transforming a current digital model of a patient's dental arch using a prior treatment plan having a plurality of stages, the method comprising:
using tooth numbering to form, for each stage of the prior treatment plan, matching pairs of teeth of the prior treatment plan and the current digital model, wherein the prior treatment plan comprises a plurality of stages that each include a digital model of a dental arch configuration having a plurality of numbered teeth, further wherein the current digital model of the patient's dental arch comprises a plurality of numbered teeth;
rejecting each matching pair if a quality of match is below a threshold;
setting, for each matched pair, one or more reference points on a surface of each tooth of the prior treatment plan and mapping the one or more reference points to the surface of each tooth of the current digital model of the matched pair;
comparing, for each stage, an arrangement of the matched pairs of teeth using the one or more reference points to identify which stage of the plurality of stages of the prior treatment plan best matches the current digital model, wherein comparing the arrangement of the matched pairs of teeth comprises generating a transformation between a set of reference points for each stage and a set of reference points for the current digital model;
transforming the stage of the plurality of stages of the prior treatment plan that best matches the current digital model using the transformation between the set of reference points for each stage and a set of reference points for the current digital model to form a new current digital model; and
outputting the new current digital model.

28.-43. (canceled)
